(19) United States
(12) Patent Application Publication
(10) Pub. No.: US 2008/0085512 A1
(43) Pub. Date: Apr. 10, 2008

(54) Array assay devices and methods for making and using the same

(76) Inventors: Petula N. D'Andrade, Loveland, CO (US); Eric M. Leproust, Loveland, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/544,395

(22) Filed: Oct. 5, 2006

(51) Int. Cl. C12Q 1/68; G01N 33/53; C12M 3/00

(52) U.S. Cl. 435/6; 435/7.1; 435/287.2; 427/2.11

(57) ABSTRACT

Aspects of the invention include methods for producing a backing element for use in an array assay device. An embodiment of the invention includes providing a precursor backing element, e.g., a solid support having a fluid retaining structure on a planar surface thereof, subjecting the precursor backing element to a solvent/plasma treatment step and then contacting the solvent/plasma treated backing element with a surface energy modification agent. Also provided are backing elements produced in accordance with embodiments of the invention, as well as array assay devices that include the backing elements. In addition, methods of using the array assay devices in analyte detection applications, as well as kits for use therein are provided.

Microarray assay protocols that employ addressable arrays of chemical agents, such as nucleic acids and polypeptides, are employed in a variety of different applications, including gene expression analysis, proteomic analysis, comparative genomic hybridization analysis, location analysis applications and miRNA applications. In such applications, an addressable array of different chemical agents, e.g., nucleic acids, is contacted with a sample of interest. Resultant binding complexes on the surface of the array are then detected to determine whether one or more analytes of interest is present in the sample.

A variety of different formats have been developed for performing microarray assay protocols. One format that has been developed is where an array element having one or more distinct arrays displayed on a surface of a planar solid support is mated to a backing element. The backing element has a fluid retaining structure, e.g., a gasket, displayed on a surface of a planar solid support. When the array element and backing element are mated to each other, a sealed volume is produced over the array(s) on the array element. Sample is conveniently introduced into and/or removed from the sealed volume during use in a number of different ways, e.g., by using sample introduction/removal ports, by placing the sample on the backing element prior to mating with the array element, etc.

### SUMMARY

Aspects of the invention include methods for producing a backing element for use in an array assay device. An embodiment of the invention includes providing a precursor backing element, e.g., a solid support having a fluid retaining structure on a planar surface thereof, subjecting the precursor backing element to a solvent/plasma treatment step and then contacting the solvent/plasma treated backing element with a surface energy modification agent. Also provided are backing elements produced in accordance with embodiments of the invention, as well as array assay devices that include the backing elements. In addition, methods of using the array assay devices in analyte detection applications, as well as kits for use therein are provided.

### BRIEF DESCRIPTION OF THE DRAWINGS

FIG. 1 illustrates a solid support carrying a microarray, such as may be used in accordance with the methods of the present invention.

FIG. 2 is an enlarged view of a portion of FIG. 1 showing multiple ideal spots or features.

FIG. 3 is an enlarged illustration of a portion of the solid support in FIG. 2.

FIG. 4 illustrates an exemplary embodiment of a backing element according to an embodiment of the invention.

FIGS. 5A-5C illustrate an exemplary embodiment of a backing element produced in accordance with embodiments of the invention, operatively positioned with respect to an array element of FIG. 1 to produce an array assay device.

### DEFINITIONS

The term “polymer” means any compound that is made up of two or more monomeric units covalently bonded to each other, where the monomeric units may be the same or different, such that the polymer may be a homopolymer or a heteropolymer. Representative polymers include polypeptides, polysaccharides, nucleic acids and the like, where the polymers may be naturally occurring or synthetic.

The term “oligomer” is used herein to indicate a chemical entity that contains a plurality of monomers. As used herein, the terms “oligomer” and “polymer” are used interchangeably, as it is generally, although not necessarily small “polymers” that are prepared using the functionalized solid supports in accordance with the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In certain embodiments, oligomers range from about 2-50 monomers, such as from about 3 to about 20, and including from about 4 to about 10 monomers.

The term “ligand” as used herein refers to a moiety that is capable of covalently or otherwise chemically binding a compound of interest. The arrays of solid support-bound ligands produced by the methods in accordance with the invention can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the solid support surface.

The term “peptide” as used herein refers to any compound produced by amide formation between carboxyl group of one amino acid and an amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length

The term “polynucleotide” as used herein refers to single- or double-stranded polymers composed of nucleotide monomers of greater than about 100 nucleotides in length.

The term “array” encompasses the term “microarray” and refers to an ordered distribution of chemical features, e.g., polymeric features (such as polynucleotides, peptide, nucleic acids and the like), on a solid support surface.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base). Synthesis of nucleic acids of this type utilizes an initial solid support-bound monomer that may be used as a building-block in a multi-step synthesis procedure to form a complete nucleic acid.

The term “array,” refers to any one-dimensional, two-dimensional or substantially two-dimensional (as well as a three-dimensional) arrangement of addressable regions (i.e., features) bearing a particular chemical moiety or moieties (such as ligands, e.g., biopolymers such as polynucleotide or oligonucleotide sequences (nucleic acids), polypeptides (e.g., proteins), carbohydrates, lipids, and the like) associated with that region.

As such, an “addressable array” includes any one or two or even three-dimensional arrangement of discrete regions (or “features”) bearing particular biopolymer moieties (for example, different polynucleotide sequences) associated with that region and positioned at particular predetermined locations on the solid support (each such location being an “address”). These regions may or may not be separated by intervening spaces. Arrays of interest include arrays of polymeric binding agents, where the polymeric binding agents may be any of: polypeptides, proteins, nucleic acids, polysaccharides, synthetic mimetics of such biopolymeric binding agents, and the like. In certain embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but may be attached at one of their termini (e.g. the 3′ or 5′ terminus). Sometimes, the arrays are arrays of polypeptides, e.g., proteins or fragments thereof.

Any given solid support may carry one, two, four or more arrays disposed on a front surface of the substrate. Depending upon the intended use, any or all of the arrays may be the same or different from one another and each may contain multiple spots or features. An array may contain more than ten, more than one hundred, more than one thousand or more than ten thousand features, or even more than one hundred thousand features, in an area of less than 20 cm² or even less than 10 cm². Features may have widths (that is, diameter, for a round spot) in the range from about 0.10 μm to about 1.0 cm. In other embodiments each feature may have a width in the range of about 1.0 μm to about 1.0 mm, such as from about 5.0 μm to 500 μm, and including from about 10 μm to about 200 μm. Non-round features may have area ranges equivalent to that of circular features with the foregoing width (diameter) ranges.

In certain embodiments, at least some, or all, of the features are of different compositions (for example, when any repeats of each feature composition are excluded the remaining features may account for at least 5%, 10%, or 20% of the total number of features). Interfeature areas may be present which do not carry any ligand. Such interfeature areas may be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than about 100 cm², or even less than about 50 cm², about 10 cm² or about 1 cm². In certain embodiments, the solid support carrying the one or more arrays is shaped as a rectangular solid (although other shapes are possible), having a length of more than about 4 mm and less than about 1 m, such as more than about 4 mm and less than about 600 mm, including less than about 400 mm; a width of more than about 4 mm and less than about 1 m, such as less than about 500 mm, e.g., less than about 400 mm; and a thickness of more than about 0.01 mm and less than about 5.0 mm, such as more than about 0.1 mm, e.g., less than about 2 mm and including more than about 0.2 and less than about 1 mm. With arrays that are read by detecting fluorescence, the solid support may be of a material that emits low fluorescence upon illumination with the excitation light. Additionally, the solid support may be relatively transparent to reduce the absorption of the incident illuminating laser light and subsequent heating if the focused laser beam travels too slowly over a region. For example, a solid support may transmit at least 20%, or 50% (or even at least 70%, 90%, or 95%), of the illuminating light incident on the front surface as may be measured across the entire integrated spectrum of such illuminating light or alternatively at 532 nm or 633 nm.

Arrays may be fabricated using drop deposition from the modified pulse jet heads of the invention. The arrays may be fabricated of either precursor units (such as nucleotide or amino acid monomers) in the case of in situ fabrication, or of a previously obtained biomolecule, e.g., polynucleotide or polypeptide. Such methods are described below and may include aspects of the methods described in, for example, U.S. Pat. Nos. 6,242,266; 6,232,072; 6,180,351; 6,171,797; and 6,323,043; as well as U.S. patent application Ser. No. 09/302,898, and the references cited therein, with the appropriate modifications being made to the fluid dispensing heads and their methods of use, in accordance with the methods of the instant invention. Other drop deposition methods can be used for fabrication.

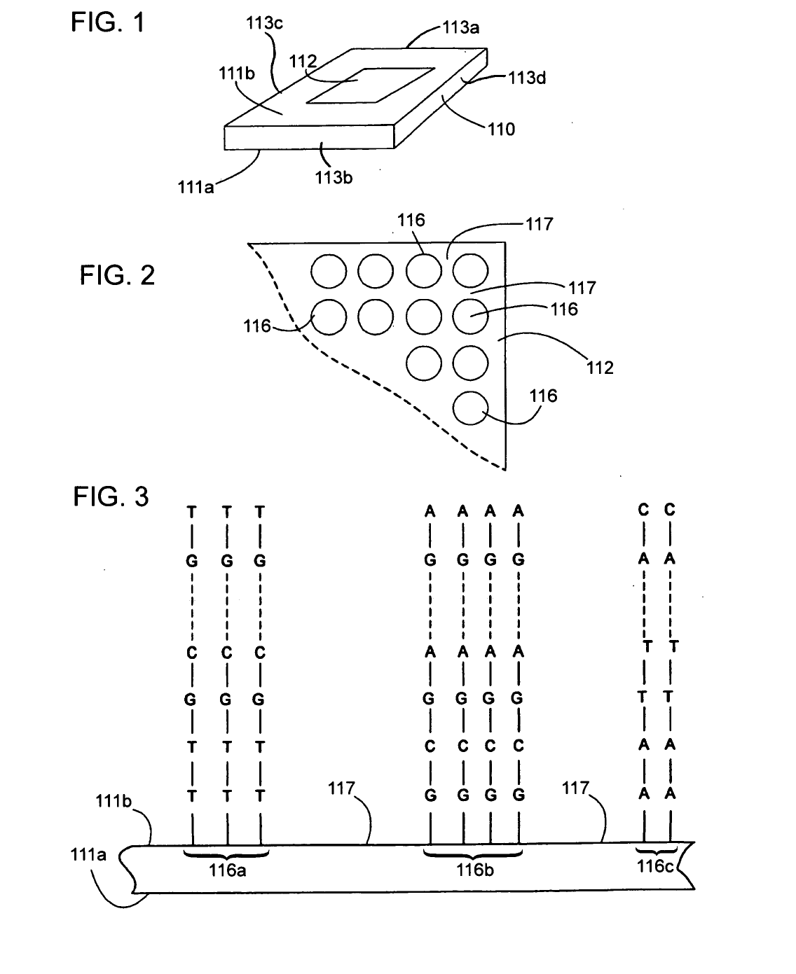

An exemplary chemical array is shown in FIGS. 1-3, where the array shown in this embodiment includes a contiguous planar solid support (also referred to herein as a substrate) 110 carrying an array 112 disposed on a rear surface 111b of solid support 110. It will be appreciated though, that more than one array (any of which are the same or different) may be present on rear surface 111b, with or without spacing between such arrays. That is, any given solid support (e.g., substrate) may carry one, two, four or more arrays disposed on a front surface of the solid support and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of support 110, not being covered by any array 112. A front surface 111a of the support 110 does not carry any arrays 112. Array 112 can be designed for testing against any type of sample, whether a trial sample, reference sample, a combination of them, or a known mixture of biopolymers such as polynucleotides. Solid support 110 may be of any shape, as mentioned above.

As mentioned above, array 112 contains multiple spots or features 116 of biopolymers, e.g., in the form of polynucleotides. As mentioned above, all of the features 116a-c may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Solid support 110 may carry on front surface 111a, an identification code, e.g., in the form of bar code (not shown) or the like printed on a solid support in the form of a paper label attached by adhesive or any convenient means. The identification code contains information relating to array 112, where such information may include, but is not limited to, an identification of array 112, i.e., layout information relating to the array(s). The support may be porous or nonporous. The support may have a planar or non-planar surface.

In those embodiments where an array includes two or more features (e.g., 16a-16c) immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features may be separated by intervening spaces. In the case of an array, the “target” will be referenced as a moiety in a mobile phase (for instance, a fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An “array element” includes a solid support and at least one chemical array, e.g., on a surface the solid support. Array elements may include one or more chemical arrays present on a surface of a solid support structure that includes a pedestal supporting a plurality of prongs, e.g., one or more chemical arrays present on a surface of one or more prongs of such a device. An array element may include other features (such as a housing with a chamber from which the substrate sections can be removed).

The terms “Array Assay Device” and “Hybridization chamber” are used interchangeably to refer to a “backing element/microarray assembly structure that at least includes (1) a backing element having a fluid retaining structure (e.g., a gasket), and (2) an array element (i.e., microarray assembly) operatively joined together so that the fluid retaining structure is positioned between the backing element solid support and microarray solid support about at least one microarray of the microarray assembly to provide a sealed microarray (e.g., hybridization) chamber about at least one array of the microarray assembly. The fluid retaining structure may be fixedly or securely attached (e.g., using adhesives, form in place processes, and the like) to the backing element solid support or the microarray solid support such that it is not readily removable therefrom or the fluid retaining structure may be a separate component.

The term “solid support” as used herein refers to a surface upon which another element, e.g., an array or fluid retaining structure may be adhered. A solid support may be configured as a substrate. Glass slides are the most common substrate for biochips, although fused silica, silicon, plastic and other materials are also suitable.

The term “sample” as used herein relates to a material or mixture of materials, for instance, in fluid form, containing one or more components of interest.

“Fluid” is used herein in its conventional sense to denote either a gaseous or liquid phase.

“Hybridizing” and “binding”, with respect to polynucleotides, are used interchangeably.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant about 5-fold more, such as less than about 3-fold more. Other stringent hybridization conditions may also be employed, as appropriate.

“Contacting” means to bring or put together. As such, a first item is contacted with a second item when the two items are brought or put together, e.g., by touching them to each other.

“Depositing” or “dispensing” means to position (i.e., place) an item at a location-or otherwise cause an item to be so positioned or placed at a location. Depositing includes contacting one item with another. Depositing or dispensing may be manual or automatic, e.g., “depositing” an item at a location may be accomplished by automated robotic devices. As used herein, “depositing a solid activator” at a location encompasses depositing or dispensing a fluid composition comprising activator at a location and removing fluid from the composition so that the solid activator remains.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “chamber” references an enclosed volume (although a chamber may be accessible through one or more ports). It will also be appreciated that throughout the present application, that words such as “top,” “upper,” and “lower” are used in a relative sense only. Chambers may include elements for material ingress or egress, e.g., sample introduction/removal ports, which ports may be sealable, e.g., comprise a septum.

“Optional” or “optionally” means that the subsequently described circumstance may or may not occur, so that the description includes instances where the circumstance occurs and instances where it does not.

It will be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

### DETAILED DESCRIPTION

Aspects of the invention include methods for producing a backing element for use in an array assay device. An embodiment of the invention includes providing a precursor backing element, e.g., a solid support having a fluid retaining structure on a planar surface thereof, subjecting the precursor backing element to a solvent/plasma treatment step and then contacting the solvent/plasma treated backing element with a surface energy modification agent. Also provided are backing elements produced in accordance with embodiments of the invention, as well as array assay devices that include the backing elements. In addition, methods of using the array assay devices in analyte detection applications, as well as kits for use therein are provided.

Before the present invention is described in greater detail, it is to be understood that this invention is not limited to particular embodiments described herein. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood in the art to which this invention belongs. Although any methods and materials similar or equivalent to those described herein can also be used in the practice or testing of the present invention, the methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope of the present invention. Any recited method can be carried out in the order of events recited or in any other order which is logically possible.

In further describing various aspects of the invention, methods for producing array assay device backing elements in accordance with embodiments of the invention are reviewed first in greater detail. Next, a review of array assay devices having backing elements produced in accordance with methods of invention, as well as applications in which array assay devices may be employed, is provided.

### Methods

As summarized above, the subject invention provides methods for producing an array assay device backing element. Aspects of the methods include subjecting a backing element precursor structure to a solvent/plasma treatment step, followed by modifying at least a portion of the solvent/plasma treated backing element with a surface energy modification agent. In further describing aspects of the methods, a non-limiting review of backing elements that may employed in the methods is first provided, followed by a review of embodiments of methods of treating the backing elements.

### Backing Elements

Backing elements that may be employed in the subject methods may vary, and in certain embodiments include a solid support having a planar surface, where a fluid retaining structure, e.g., in the form of a gasket, is present on the planar surface. Suitable solid supports can have a variety of shapes, sizes, forms and compositions and can be derived from naturally occurring materials, naturally occurring materials that have been synthetically modified, or synthetic materials. Examples of suitable support materials include, but are not limited to, nitrocellulose, glasses, silicas, teflons, and metals (for example, gold, platinum, and the like). Suitable materials also include polymeric materials, including plastics (for example, polytetrafluoroethylene, polypropylene, polystyrene, polycarbonate, and blends thereof, and the like), polysaccharides such as agarose and dextran, polyacrylamides, polystyrenes, polyvinyl alcohols, copolymers of hydroxyethyl methacrylate and methyl methacrylate, and the like. A solid support may be homogenous or a composite structure of two or more different materials, e.g., where the solid support includes a first base material that is coated on a surface with one or more additional different coating materials. For example, the solid support may be a composite that is made of a glass base layer which is coated with a metal surface layer, such as gold, or coated with a polymeric layer, such as polypropylene.

A solid support of the invention may have any desired configuration. As such, the solid support may be a uniform solid support, e.g., a wafer of solid material, such as silicon, glass, quartz, polymerics, and the like; a large rigid sheet of solid materials, e.g., glass, quartz, plastics, such as polycarbonate, polystyrene, and the like, or can comprise additional elements, e.g., structural, compositional, and the like. A flexible solid support, such as a roll of plastic as polyolefins, polyamide, and others, a transparent solid support, or combinations of these features can be employed. The solid support may include other structural elements that are part of the ultimately desired device (e.g., a hybridization chamber). Particular examples of such elements include gaskets, microcantilevers, pits, wells, posts, stand-offs, and the like.

The backing element is configured (e.g., sized, shaped) to be operatively associated or joined with an array element that has at least one array thereon to provide an array assay device. The term “backing element” is meant broadly to refer to any suitable solid support that may be operatively mated or joined with an array element to provide an array assay device that includes an assay chamber about an array of the array element.

The particular shape of a backing element may be dictated at least in part by the solid support of the microarray assembly with which the backing element may be associated. The shape of the solid support of the backing element may range from simple to complex and may be square, rectangular, oblong, oval, circular in shape, and the like.

In certain embodiments, the backing element may have a rectangular shape, and may have a length that may range from about 1 mm to about 1 m, for instance, from about 2 mm to about 600 mm, such as about 4 mm to about 400 mm, e.g., the length may range from about 25 mm to about 150 mm, for instance, from about 50 mm to about 100 mm, including about 65 mm to about 80 mm. The backing element may have a width that may range from about 1 mm to about 1 m, for instance from about 2 mm to about 500 mm, such as about 4 mm to about 400 mm, e.g., the width may range from about 15 mm to about 40 mm, for instance, from about 20 mm to about 35 mm, including from about 20 mm to about 30 mm. The backing element may have a thickness that may range from about 0.01 mm to about 5.0 mm, for instance, from about 0.02 mm to about 2 mm, including about 0.1 mm to about 1.5 mm or about 0.5 mm to about 1.5 mm. Shapes other than rectangular may have analogous dimensions. In certain embodiments, at least one surface of a backing element may be planar, but in certain embodiments, a surface may deviate from planar, e.g., portions of the surface may be non-planar (e.g., may include recessed structures, elevated structures, channels, orifices, guides, and the like).

In certain embodiments, the solid support of the backing element may be in the form of a substrate and/or wafer that includes a flat, planar surface on which a fluid retaining structure, such as a gasket, may be associated. For instance, the backing element may include a surface to which one or more fluid retaining structures may be fixedly or separably associated. In certain embodiments, a plurality of fluid retaining structures may be present on the surface of the backing element, such that a plurality of fluids (e.g., samples) may be retained in each of the fluid retaining structures present without cross contamination of the fluids.

When the backing element containing one or more fluid retaining structures is operatively joined together with an array element, an array assay device is produced that includes a tightly sealed array assay chamber. The backing element is dimensioned to fit with both a solid support carrying an array (i.e., an array element) and a fluid retaining structure positioned there between, so as to produce a reaction volume bounded on the top and bottom by the backing element surface and the array element and on the sides by the walls of the fluid retaining structure of the backing element, such that an array is contained within the area bounded by the fluid retaining structure.

By “fluid retaining structure” or “fluid retention element” is meant a structure or element that is associated with the surface of a solid support (e.g., a planar surface of the backing element) in such a manner as to retain a fluid on the surface of the solid support within an area at least partially bounded by the fluid retaining structure. Accordingly, the fluid retaining structure is interposed between the surface of a backing element and a surface an array element and thereby functions to retain a fluid between the two surfaces.

A fluid retaining structure may completely bound a surface of a solid support or may partially bound the surface, wherein the surface may be additionally bounded by one or more other structures. Hence, the fluid retaining structure forms at least a partial boundary between the opposing surface of the microarray backing element and microarray assembly. While in the broadest sense, the fluid retaining structure may be fixedly attached to the surface of a backing element, an array element, or may be a separable component, for convenience of description in the present application, the fluid retaining structure is described in the present application as part of the backing element.

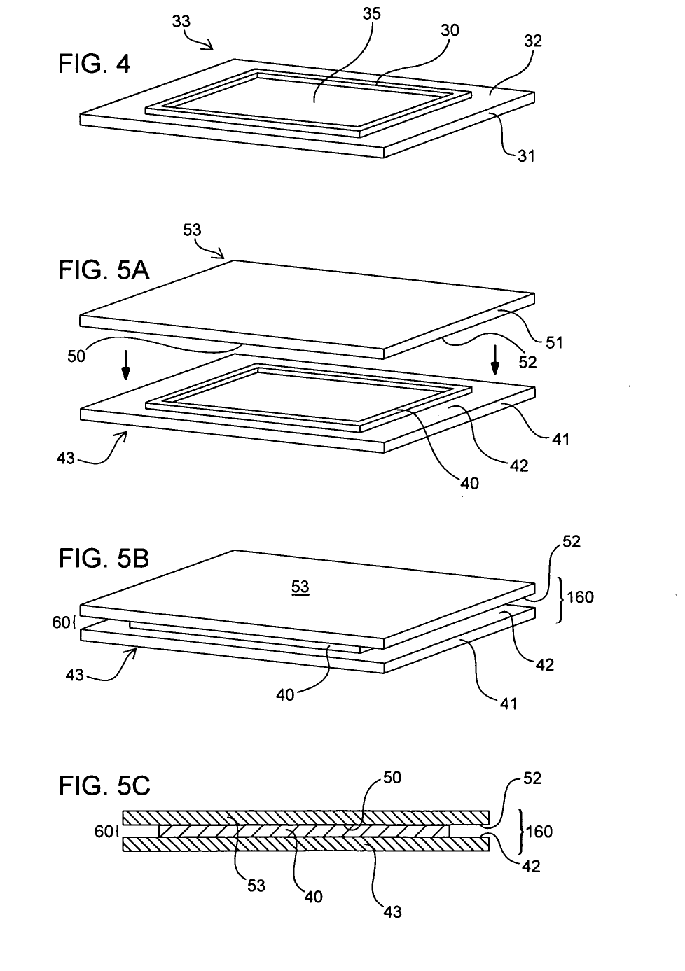

FIG. 4 shows an exemplary embodiment of a backing element 33 that may be produced in accordance with the methods of the subject invention. As shown, a microarray backing element 33 includes fluid retaining structure 30 which is disposed around and marks the perimeter of an interior area 35 on a planar surface 32 of a solid support 31.

The interior area and the fluid retaining structure thus defines a well structure that is adapted for retaining a fluid, where the well is defined by the walls of the fluid retaining structure and the planar surface of the backing element that is bounded or enclosed by the fluid retaining structure (i.e., the interior area). Specifically, a well formed by the fluid retaining structure (e.g., defined by the surface of the solid support on which the fluid retaining structure is positioned and the fluid barrier walls), may confine a liquid volume of at least about 1-5 μl, where the volume may range from about 1 μl to about 1000 μl, for instance, from about 10 μl to about 1000 μl, where the volume may be as great as about 1000 μl to about 5000 μl or greater. The shape of the interior area (e.g. of the well) may be altered depending on the desired use, e.g., by altering the configuration of the fluid retaining structure(s) and/or support surface, and the like.

The shape and configuration of a fluid retaining structure will depend on a variety of factors such as the particular array feature(s) or spot(s) the interior area of the fluid retaining structure is intended to encompass; the number, size and shape of fluid retaining structures to be present; the size, shape and pattern of the arrays included on the micro array assembly to which the fluid retaining structures are to be joined, etc. As such, the subject fluid retaining structure(s) may assume a variety of different shapes from simple to complex.

In certain embodiments, the fluid retaining structure may have a square, rectangular, oblong, oval, circular or other geometric shape. In certain embodiments, the width or diameter of a fluid retaining structure may not be constant throughout the entire thickness or height of the structure, i.e., the width or height may vary. Accordingly, shapes such as cone-like, spiral, helical, pyramidal, parabolic or frustum are possible as well. In certain embodiments, a plurality of fluid retaining structures is present, for instance, in a pattern, such as a grid or other analogous geometric or linear pattern or the like. In certain embodiments the fluid retaining structures may be present in a non grid-like or non-geometric pattern.

The number of fluid retaining structures present on the surface of the solid support or backing element may range from about 1 to about 20 or more; for example, 25 or more fluid retaining structures may be present on a single backing element. In those embodiments having more than one fluid retaining structures, it is to be understood that the dimensions (and/or the shapes and/or materials) of the fluid retaining structures may be the same or some or all of the fluid retaining structures may have different dimensions (and/or shapes and/or materials).

The physical dimensions of a fluid retaining structure may be characterized in terms of thickness (height), and/or width, and/or length. Thickness or height is defined as the perpendicular distance from the surface of the solid support to most distal (i.e., top) surface of the fluid retaining structure. The width of a fluid retaining structure is defined as the distance from one side of a fluid retaining structure through the fluid retaining structure to the opposing side of the fluid retaining structure, proceeding on a line parallel to the fluid retaining structure surface, but perpendicular to the fluid retaining structure's long axis at the particular point where the length is being measured. The length is defined as the long axis of the fluid retaining structure that is parallel to the plane of the substrate surface. In a structure having round or round-like (e.g., oblong) shapes, the length may be analogous to a major axis.

The thickness or height of a fluid retaining structure is of a dimension that is suitable to retain a sufficient amount of sample for an array assay. Accordingly, a fluid retaining structure may have a height or thickness of at least about 5 to about 100 micrometers, for instance, at least about 10 micrometers to about 50 micrometers, including at least about 15 to at least about 30 micrometers. In certain embodiments the height may be about 25 micrometers to about 100 micrometers or more, for instance, up to about 250 micrometers or more, such as up to about 500 micrometers or more. In certain embodiments the height may be up to about 1000 micrometers or more, for instance, up to about 5000 micrometers or more, such as a few millimeters or more.

The length of a fluid retaining structure may be at least about 20 to about 1000 micrometers, for instance, at least about 150 micrometers to about 750 micrometers or more, such as up to about 300 micrometers to about 500 micrometers or more, even up to about 1000 micrometers. The width of a fluid retaining structure may range up to about 1.5 mm, for instance, up to about 3 mm, such as up to about 6 mm. In certain embodiments the width may be at least about 20 to about 1000 micrometers or more, for instance, at least about 100 micrometers to about 700 micrometers or more, such as about 250 micrometers to about 500 micrometers or more.

The material(s) of the fluid retaining structure is selected to provide a fluid retaining structure having particular properties, e.g., suitable thickness, width, height, structure, fluid retaining properties, stability, inertness, array assay protocol compatibility, and the like. The subject fluid retaining structures may be flexible or deformable upon application of a suitable force thereto or may be rigid, i.e., not easily deformable or not deformable at all upon application of a suitable force thereto.

The selection of a fluid retaining structure material is determined relative to the intended application. Accordingly, the fluid retaining structure may be made of any suitable material. Suitable fluid retaining structure materials may be derived from naturally occurring materials, naturally occurring materials that have been synthetically modified, or synthetic materials. Suitable fluid retaining structure materials include, but are not limited to: polymers such as polypropylenes, urethanes including polyurethanes, acrylates, elastomers, silicone sealants (e.g., Loctite 5964 thermal cure silicone), polysulfides, latex, acrylic, and the like. In certain embodiments, the fluid retaining structure material is a fluoropolymer such as polytetrafluoroethylene, e.g., a Teflon® such as a liquid Teflon®, e.g., Teflon® AF which are a family of amorphous fluoropolymers provided by E.I. du Pont de Nemours and Company. In certain embodiments, the fluid retaining structure includes a polymer that is an elastomer (e.g., polyisoprene, polybutadiene, polyisobutylene, polyurethanes, and the like).

Backing elements that may be employed in methods of the invention, as well as methods for producing the same, include but are not limited to those described in published United States Patent Application publication nos. 2003-0231985 A1 and 2003-0231987 A1, the disclosures of which (e.g., pertaining to backing elements and their production and use) is incorporated herein by reference.

### Solvent/Plasma Treatment

Aspects of the invention include subjecting precursor backing elements, e.g., as described above, to a solvent/plasma treatment step in order to produce solvent/plasma treated backing elements. When a precursor backing element is subjected to a solvent/plasma treatment step in accordance with embodiments of the invention, the backing element is first contacted with a solvent, and the solvent contacted backing element is then contacted with a plasma.

The term “contact” is meant broadly to include any suitable technique of bringing the solvent and plasma in sufficient proximity to the backing element to be treated. For example, the backing element (either the entire element or a suitable portion thereof) may be submersed in a sufficient amount of a fluid, or may be flooded with the fluid to achieve contact. Alternatively, contacting may be accomplished by using a drop deposition technique (such as with a pipette), using a swab, using a syringe, or other convenient technique, which may be used in localized modification protocols, such as in embodiments where only a small portion of the backing element is to be contacted with the solvent or plasma, collectively referred to herein as a “modification agent.” In those embodiments where the modification agent is a gaseous agent, such as plasma, the entire member may be positioned in a plasma reactor or chamber such that the entire member is contacted with plasma or a more localized technique may be employed. In certain embodiments, areas of a backing element not intended to be contacted may be “masked” or covered to prevent the modification agent from contacting or otherwise affecting the masked area.

Any portion or all of the backing element may be modified. Accordingly, modification protocols include “global” and “local” modification. In other words, modification of the backing element may be “global” such that the entire structure member may be modified, e.g., contacted with a modification agent, or “localized” such that only a specific area of the structure member may be modified, e.g., contacted with a modification agent.

For the solvent contact step, the solvent may be any suitable solvent. In certain embodiments, the surface of the solid support (e.g., backing element) is contacted with one or more organic solvents. A variety of organic solvents may be used and include polar and non-polar organic solvents. For example, polar organic solvents that may be employed include, but are not limited to: alcohols, e.g., methanol, ethanol, isopropanol, and the like; ketones, e.g., methyl ethyl ketone, acetone, and the like; trialkyl amines, e.g., trietylamine and the like; tributyl amines; and various aromatic and cyclic polar solvents such as pyrolidinone and the like. Non-polar organic solvents that may be employed include, but are not limited to, aliphatic hydrocarbons, e.g., hexane, heptane, and the like; aromoatic hydrocarbons, e.g., toluene, benzene, xylene, cyclohexane, and the like; methyl, ethyl, and other ethers; glymes including diglyme, triglyme, and the like. Fluorosilicone compounds may also be used.

In certain methods of the invention, the solvent is a non-polar organic solvent such as hexane, heptane, toluene, benzene, xylene, cyclohexane, methyl, ethyl, glyme, diglyme, tryglyme or the like. In certain embodiments, the non-polar solvent is toluene.

The solvent may be contacted with the solid support (e.g., backing element) in any suitable manner. For instance, in certain embodiments, the solid support may be soaked in the solvent. For example, the solid support may be positioned within a suitable chamber or wash tank and then the chamber or tank may be filled with a suitable solvent, such as a non-polar organic solvent (e.g., toluene), so as to cover the backing element. In certain embodiments, the chamber is a flow cell and a quantity of the solvent is flowed over the surface of the solid support.

The amount of solvent employed may vary depending on the particular solvent employed, the surface area to be modified, etc. For example, the amount of solvent used may range from about 0.005 ml/mm² of backing element contacted to about 0.15 ml/mm² of backing element contacted, for instance, from about 0.008 ml/mm² to about 10 ml/mm² of backing element contacted, including from about 0.01 ml/mm² to about 0.05 ml/mm² of backing element contacted. The amount of time the solvent is in contact with the backing element may range from about 1 minute to about 1 week or longer, such as from about 1 minute to about 24 hours, including from about 1 minute to about 8 hours, for instance, from about 5 minutes to about 6 hours, such as from about 15 minutes to about 4 hours, including about 30 minutes to about 2 hours.

Once contacted with a suitable amount of solvent, the solid support is dried. The solid support may be dried by any suitable protocol, for instance, the solid support may be dried by air drying, nitrogen drying, vacuum drying or the like, in a manner sufficient to remove any solvent present on the surface of the solid support. The solid support may be dried for any sufficient amount of time, for instance, for about 15 to about 60 minutes, for instance, for about 20 minutes to about 40 minutes, including about 25 minutes. The solid support may further be dried by the application of heat.

The solvent may be contacted with the backing element in one or more steps. Accordingly, in certain embodiments, the dried surface of the solid support may then be contacted with another solvent, e.g., a clean (“fresh”) second organic or inorganic solvent, which may be the same type of solvent previously used or a different type of solvent. The solid support may be contacted with the fresh solvent for a desired period of time, such as a period of time ranging from 1 minute to about 1 week, including from about 1 minute to about 1 day, including from about 5 minutes to about 90 minutes. Once contacted for the appropriate length of time, the solid support may then be dried, as described above.

In one embodiment, a backing element that includes a glass or silica solid support (e.g., a substrate) and one or more elastomeric gaskets, may be positioned in a suitable carrier for easy handling, such as a stainless steel carrier that will not degrade upon contact with a solvent. The carrier with the backing element may then be positioned in a suitable, empty solvent tank. Once the backing element is positioned in the tank, the tank may be filled with a suitable volume of solvent (e.g., toluene) such that the backing element is covered by the solvent, e.g., a sufficient amount of solvent is such that the solvent level is about ¼ inch or more above the topmost surface of the backing element as it is positioned in the tank.

The duration of contact between the backing element and the solvent (e.g., toluene) may range from about 1 hour (or shorter) to about 2 hours (or longer), e.g., one hour with agitation (e.g., rocking, stirring, and the like) of the tank every fifteen minutes or two hours with agitation of the tank at the end of two hours. After contact with the first volume of solvent, the tank is emptied of solvent. The tank may then be filled with a clean or fresh volume of solvent in a manner analogous to that described above. Contact duration of the backing element with the second volume of solvent may be about thirty minutes with suitable agitation of the tank, e.g., agitation of the tank about every ten minutes. The tank may then be emptied of the second volume of solvent and the carrier with backing element removed. The backing element may be air dried for about one hour or more to remove any remaining solvent from the backing element.

Aspects of the invention further include subjecting a solvent treated backing element to a plasma. Although, any suitable plasma may be used, in certain embodiments of the invention, the plasma is an ionized gas. The plasma may be a gas such as air, nitrogen, oxygen, nitrous oxide, helium, water vapor, carbon dioxide, methane, a noble gas, or a combination thereof.

Plasma modification of the solvent treated solid support (e.g., backing element) may be performed in any suitable manner. For instance, as described below, a solid support may be contacted with plasma within a suitable plasma reactor. Such reactors may include a vacuum chamber that includes a vacuum pump, purge element, process gas sources and regulators, a source of energy for gas ionization (such as electromagnetic energy), and may additionally include a microprocessor(s), software, circuitry, etc. for automatically implementing and controlling the process parameters for plasma modification, such as the time, gas flow, and amount of energy to be applied. Accordingly, the backing element may be positioned within a radio-frequency (RF) plasma chamber and a gaseous (e.g., oxygen) atmosphere may be introduced so as to modify (e.g., oxidize) a surface of the solid support.

While the exact parameters of a plasma protocol may vary depending on the gas employed, modification desired, etc., in certain embodiments, the plasma modification process according to the subject methods may include the following: First, after one or more solid supports is positioned within the reactor and the reactor is sealed, the reactor is then pumped down to a predetermined vacuum pressure (base pressure). A solid support may be loaded into the reactor chamber either alone or in combination with other solid supports. For instance, a plurality of solid supports may be loaded into one or more trays which then may be positioned within the reactor. Second, the process gas (which may be a single gas or a plurality of different gases, for instance, oxygen) is introduced and allowed to stabilize at a desired process pressure. Third, the plasma is initiated by the application of a suitable energy source, such as radio frequency (“RF”) energy. Fourth, the RF power is shut off and gas delivery is processed for a desired length of time. Fifth, the reactor is pumped down to the base pressure to eliminate residual process gas(es). Next, the reactor is vented to atmospheric pressure. Finally, the plasma modified solid support is removed from the reaction chamber.

The ionization of the one or more gases may be accomplished by providing an energy field. An energy field may be provided by any suitable energy source such as an RF generator, microwave generator, DC power generator, and the like. For example, a suitable RF generator may be employed and includes low frequency RF generators (90 KHz-1 MHz), mid-frequency RF generators (1-4 MHz), high frequency RF generators (13.56 MHz), and extended-frequency RF generators (27.12-40.68 MHz). Power may range from a few watts to kilowatts. For example, where RF is employed, RF power may range from about 20 watts to about 1000 watts, for instance, from about 50 to about 500 watts, such as from about 100 watts to about 400 watts.

The particular plasma parameters will vary depending on the particular plasma modification performed, e.g., the gas, desired modification, etc. In certain embodiments, the duration of the plasma modification may range from about 1 minute to about 60 minutes, for instance, from about 3 minutes to about 30 minutes, such as from about 5 minutes to about 25 minutes, including about 20 minutes. The temperature at which the plasma modification is performed may range from about 20° C. to about 200° C., for instance, from about 30° C. to about 150° C., such as from about 50° C. to about 130° C. or about 80° C. The power may range from about 20 watts to about 1000 watts, for instance, from about 50 watts to about 300 watts, such as from about 100 watts to about 250 watts, including about 200 Watts. The gas flow rate may range from about 0.1 ml/min to about 300 ml/min, for instance, from about 0.3 ml/min to about 200 ml/min, such as from about 10 ml/min to about 100 ml/min. The system pressure may range from about 0.01 Torr to about 20 Torr, for instance, from about 0.05 Torr to about 10 Torr, such as from about 0.1 Torr to about 1 Torr.

In certain plasma treatment steps, the surface energy of the backing element is increased, such that the surface becomes more hydrophilic. The magnitude of increase may vary, and in certain embodiments is accompanied by a decrease in contact angle, e.g., of about 5° or more, such as about 10° or more, including about 25° or more, e.g., about 35° or more, about 45° or more, about 50° or more, as determined by measuring the contact angle of the surface before and after plasma treatment and comparing the measured contact angles. In certain embodiments, following solvent/plasma treatment, the contact angle of the surface may be range from about 0 to about 40°, such as from about 0 to about 30° and including from about 10 to about 30°, where the contact angle is determined using the protocol as described in Contact Angle Measurements Using the Drop Shape Method by Roger P. Woodward, which can be obtained at the website having an address that is formed by placing “http://www.” in front of “firsttenangstroms.com/pdfdocs/CAPaper.pdf”.

### Surface Energy Modification

Once the solid support (e.g., backing element) is contacted with suitable solvent/plasma and the solvent/plasma treatment performed, in accordance with embodiments of the invention the solvent/plasma treated solid support may then be contacted with a surface energy modification agent. In certain embodiments, the backing element is contacted with a surface energy modification agent in a manner sufficient to decrease the surface energy of the surface and thereby make the surface more hydrophobic. As such, in embodiments of the invention, this step results in the surface energy of the backing element being decreased. The magnitude of decrease in surface energy may vary, and in certain embodiments is accompanied by an increase is contact angle of about 5° or more, such as about 10° or more, including about 25° or more, e.g., about 35° or more, about 45° or more, about 50° or more, as determined by measuring the contact angle of the surface before and after plasma treatment and comparing the measured contact angles.

Any convenient surface energy modification agent may be employed in this step of the methods. In certain embodiments, the surface energy modification agent is a polymeric surface energy modification agent. Of interest in certain embodiments are alkyl polysiloxane surface modification agents. Alkyl poly siloxanes of interest include, but are not limited to, those described by the general formula:

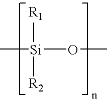

wherein:

n is any integer greater than 1 and may about 1000 or higher, e.g., 500 or higher;

R₁ is a lower alkyl radical, e.g., having from 1 to 7 carbon atoms; and

R₂ is hydrogen, a lower alkyl radical, e.g., having from 1 to 7 carbon atoms or an aryl radical containing about 6 carbon atoms.

Examples of suitable alkylpolysiloxanes include, but are not limited to: dimethyl polysiloxane, methyl hydrogen polysiloxane, and methyl phenyl polysiloxane, etc. In certain embodiments, n ranges from about 5 to about 900. The molecular weight of the polysiloxane may vary, and in certain embodiments ranges from about 400 to about 60,000.

The surface modification agent may include the alkyl polysiloxane in a suitable solvent, such that a fluid composition of an alkyl polysiloxane is employed. The fluid compositions including alkyl polysiloxanes employed may also include from about 5 to about 25 percent by weight siloxane in an appropriate solvent. Any solvent for the siloxane may be used, including economical, easy to use, low health hazards, water miscible solvents. Various solvents, diluents and extenders may also be included, such as but not limited to: alcohols, chlorinated-hydrocarbons, ethers, ketones, esters, aromatic hydrocarbons, water, colloidal pyrogenic silicas and clays. Examples of such solvents and diluents are benzene, butyl acetate, carbon tetrachloride, ethyl ether, gasoline, hexane, isopropyl alcohol, methyl ethyl ketone, mineral spirits, perchloroethylene, toluene, xylene, etc. Other solvents also may be used. It is noted that the suitability of a solvent depends in part on the particular alkyl polysiloxane used and the intended application. Alcohols are suitable solvents for use according to the present invention, such as isopropanol.

The amount of solvent to be used in a composition of the present invention can vary widely. In certain embodiments, an amount of solvent about 75-95% by weight of the final composition, such as about 80-90% by weight of the final composition, is employed.

In certain embodiments, the fluid composition further includes an acid modifier. Suitable acid modifiers include, but are not limited to: sulfuric acid, phosphoric acid, aromatic sulfonic acids, aliphatic sulfonic acids and hydrochloric acid. Although the amount of acid which can be present may vary, in certain embodiments an amount of acid equivalent to about 2.5 to 30% based on the weight of the polysiloxane in the composition is employed. Thus, if 2% dimethyl polysiloxane is employed with isopropyl alcohol as a solvent, about 1% acid based on the weight of the overall composition, or 50% based on the weight of the polysiloxane, is present in certain embodiments. In certain embodiments, an amount of acid be used which is equivalent to about 5-20% by weight of the polysiloxane, such as about 1% by weight of acid, based on the weight of the composition, is employed.

In certain embodiments, the surface modification agent that is employed is a composition sold under the trademark Rain-X®. This composition is a composition of polydimethyl siloxane dissolved in an alcohol, e.g., ethanol or isopropanol, and acidified with a few percent sulfuric acid. The product is commercially available from SOPUS Products (Houston Tex.). This composition is also described in U.S. Pat. No. 3,579,540, the disclosure of which is herein incorporated by reference.

Also of interest as surface modification agents are silanes, such as those described in U.S. Pat. Nos. 6,258,454 and 6,444,268, the disclosures of the silanes of in these applications being herein incorporated by reference.

The surface energy modification agent may be contacted with a solid support (e.g., backing element) of the invention in any suitable manner. For instance, the surface may be contacted with a surface energy modification agent via wet chemistry methods, e.g., by dipping the surface into a composition containing the surface energy modification agent. In another embodiment, the surface energy modification agent may be contacted with the surface of the solid support via a gas phase reaction process. The gas phase reaction process may include chemical or molecular vapor deposition. Chemical vapor deposition (CVD) is a generic name for a group of related processes that involve coating a surface by depositing a material from a vapor phase. In certain embodiments, molecular vapor deposition, such as the MVD process as described in published U.S. patent application 20040261703 (incorporated herein by reference in its entirety) is employed.

Any suitable CVD apparatus and protocol may be used. In certain embodiments, the CVD apparatus may include a process chamber for holding a solid support and for vapor deposition of a coating of a vapor on to a surface of the solid support, at least one container for containing a vapor precursor in liquid or solid form, at least one precursor vapor reservoir for holding a vapor of the liquid or solid precursor, vapor flow control mechanisms for controlling the flow of the precursor vapor(s) from the precursor container(s) to the precursor reservoir(s) and the flow from the precursor reservoir(s) to the process chamber, at least one pressure sensor, and a process controller for controlling the vapor deposition process.

While the exact parameters of a CVD protocol may very depending on the composition employed, modification desired, etc., in certain embodiments, the CVD process according to the subject methods may include the following: First, positioning a solid support to be modified in a suitable vapor deposition apparatus (e.g., in a vapor deposition process chamber). Second, one or more suitable liquid or solid precursors contained in one or more precursor containers is converted into the vapor phase. The vapor of the precursor(s) is then transferred to the one or more precursor vapor reservoirs in which the precursor vapor(s) accumulates. Once a suitable amount of vapor is accumulated, the vapor is then transferred to the processing chamber wherein the vapor contacts the solid support thereby coating one or more surfaces of the solid support with the vapor. Once contacted for a sufficient amount of time, the solid support is removed from the process chamber and allowed to dry by any suitable method (e.g., air drying, nitrogen drying, vacuum drying, heating, and the like).

As noted above, the particular CVD parameters will vary depending on the particular CVD modification performed, e.g., the number and type of liquid or solid precursors to be used, the desired modification, etc. However, in certain embodiments, the precursor(s) is introduced into the chamber at various flow rates to establish and maintain a pressure in the chamber ranging from about 100 mTorr to 150 Torr, for instance, from about 0.01 Torr to about 100 Torr, for instance, about 0.2 Torr to about 70 Torr, such as about 0.5 Torr to about 10 Torr, including about 3 Torr to about 5 Torr. In certain embodiments the source gas flow rate ranges from about 50 sccm to about 300 sccm, for instance, from about 100 sccm to 200 sccm, including about 100 sccm. The processing time may range from about 1 minute to about 4 hours, for instance, from about 1 minute to about 50 minutes, such as from about 5 minutes to 30 minutes. In certain embodiments, the formation of a coating on at least a portion of the surface of the solid support is carried out at a temperature ranging between about 5° C. to about 150° C., such as 20° C. to about 125° C., including about 60° C. to about 100° C. In certain embodiments, the thickness of the applied coating ranges from about 0.1 Å to about 2000 Å, for instance, from about 1 Å to about 1000 Å, such as 5 Å to about 500 Å, including about 300 Å.

In certain embodiments, treatment with the surface modification agent results in the production of a backing element having uniformly hydrophobic surface of low surface energy and a high contact angle. In certain embodiments, following solvent/plasma treatment, the contact angle of the surface may be in the range from about 0 to about 500, such as from about 5 to about 30° and including from about 10 to about 20°.

At any point in time during or after one or more modifications of a surface of a solid support, the solid support may be inspected for quality control, for example, for information regarding a particular parameter to be monitored. The particular parameter to be measured may be any relevant parameter that may affect the function and/or quality of the solid support end product. For instance, during the fabrication of a solid supports, the solid supports may be selected for under going one or more quality control measurements, wherein a specified limited variability of a given parameter is measured. In certain embodiments, the given parameter is the hydrophobicity or hydrophilicty of the surface of the solid support wherein the contact angle is measured to determine if the surface of the solid support has a contact angle that falls within a specified variability range, for instance within 100 of a predetermined contact angle. In this manner, the hydrophobicity and/or hydrophilicity of the solid support can be determined and if the value is outside of an acceptable range the solid support may be reprocessed or discarded.

It is to be noted that where a plurality of solid supports are fabricated together, for instance, in a batch, one or more solid supports may be selected for one or more quality control measurements so as to determine the quality of the entire batch. In this manner, a plurality of solid supports may be fabricated and modified in accordance with the methods of the invention while at the same time the quality of the manufacturing process can be closely monitored and controlled so as to ensure that the manufactured solid supports fall within a specified quality range. It is to be noted that although the above has been described with reference to a predetermined hydrophobicity and/or hydrophilicity value as measured by the contact angle of a surface of a solid support, any relevant parameter or physical characteristic of the solid support and/or fluid retaining structure may be measured by any suitable methods to control the quality of the manufactured solid supports, gaskets, assay chambers and the like.

Embodiments of the invention result in the manufacture of highly uniform backing elements. In certain embodiments, the backing elements are highly uniform with respect to surface energy properties, both within a given batch and from batch to batch. As such, any difference in surface energy properties, in terms of contact angle, between two different backing elements produced in the same or even different batches will not vary by more than about 10°, and in certain embodiments will not vary by more than about 5° or even more than about 2°, e.g., 1°.

### Array Assay Devices and Applications Thereof

Aspects of the invention further include array assay devices that include backing elements produced in accordance with embodiments of the invention, e.g., as described above. Array assay devices of the invention include an array element, e.g., comprising a solid support that contains a surface having at least one array of chemical entities disposed thereon, and a backing element. The array element of the array assay devices includes an addressable array, e.g., as described above. The addressable array may be produced using any convenient protocol. For example, microarray probes may either be synthesized directly on the microarray solid support or attached to the solid support after they are made. Arrays can be fabricated using drop deposition from pulsejets of either polynucleotide or polypeptide precursor units (such as monomers) in the case of in situ fabrication, or previously obtained pre-made polynucleotides or polypeptides may be deposited on to the surface of the solid support. Such methods are described in detail in, for example, in U.S. Pat. No. 6,242,266, U.S. Pat. No. 6,232,072, U.S. Pat. No. 6,180,351, U.S. Pat. No. 6,171,797, U.S. Pat. No. 6,323,043, and the references cited therein (all of which are hereby incorporated by reference).

FIGS. 5A-5C show an exemplary embodiment of the elements of an array assay device 160, where a backing element produced in accordance with aspects of the invention is employed. FIGS. 5A-5C are described with reference to a backing element containing a fluid retaining structure (e.g., a gasket) for exemplary purposes only and is in no way intended to limit the invention.

As shown in FIGS. 5A-5C, a process for providing a sealed assay chamber (e.g., a hybridization chamber) in an array assay device is presented. In one step, a backing element 43 is provided. The backing element contains at least one gasket 40 that is positioned on a surface 42 of the solid support 41 of the backing element 43. In another step, an array element 53 having a solid support 51 containing one or more arrays 50 (not shown) on a surface 52 of the support 51 is provided. Next, as seen with reference to FIG. 5A, the backing element 43 is positioned in opposition to the array element 53. The backing element 43 is positioned such that gasket 40 of backing element 43 is facing and is in direct opposition to the surface 52 of the solid support 51 of the array element. Next, the backing element 43 and array element 53 are carefully brought into increasingly close proximity to one another so as to “sandwich” the gasket 40 between the backing element 43 and array element 53, as shown in FIG. 5B and FIG. 5C, where FIG. 5C shows a cross sectional view of the operatively positioned backing element and array element of FIG. 5B. In this manner, an array assay device 160 is provided that forms a sealed array assay chamber 60 about the one or more arrays 50 by surface 52 of array element 53, surface 42 of backing element 43 and the walls of the gasket 40.

The array assay devices of the invention may be employed in a variety of different array assay protocols. Any suitable protocol for carrying out such assays may be employed. For instance, a sample suspected of containing an analyte of interest may be contacted with an array within an array assay chamber of an array assay device, e.g., as described above, under conditions sufficient for the analyte to bind to its respective binding pair member (i.e., probe) that is present on the array. If the analyte of interest is present in the sample, it will bind to the array at the site of its complementary binding member and a complex will formed on the array surface. The presence of this binding complex on the array surface may then detected, e.g., through use of a signal production system, e.g., an isotopic or fluorescent label present on the analyte. The presence of the analyte in the sample may then be deduced from the detection of binding complexes on the surface of the microarray assembly. Sample may be introduced into the chamber using any convenient protocol, e.g., via a sample introduction port, etc.

Analyte detection applications of interest include, but are not limited to, hybridization assays in which a hybridization chamber containing one or more nucleic acid arrays of the invention is employed. In these assays, a sample of target nucleic acids is first prepared, where preparation may include labeling of the target nucleic acids with a label, e.g., a member of a signal producing system. Following sample preparation, the sample is contacted with a microarray assembly of a hybridization chamber of the invention, under hybridization conditions, whereby complexes are formed between target nucleic acids that are complementary to probe sequences attached to the surface of the microarray solid support. The presence of hybridized complexes is then detected.

Hybridization assays of interest which may be practiced using the assay chambers of the invention include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Where the arrays are arrays of polypeptide binding agents, e.g., protein arrays, specific applications of interest, which may be used in conjunction with the array assay chambers of the invention, include analyte detection/proteomics applications, including those described in: U.S. Pat. No. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of which are herein incorporated by reference.

In a specific embodiment, a backing element containing a fluid retaining structure, produced by the methods of the invention, is used in an array assay protocol. In a first step, a sample suspected of including an analyte of interest, i.e., a target molecule, is contacted with the modified backing element, to produce a supported sample, e.g., a backing element supported sample (in certain embodiments the sample may be contacted with the microarray).

A sample may be contacted with a modified backing element by depositing an amount of sample in the one or more fluid retaining structures positioned on a surface of the modified backing element to confine a certain amount of sample to a certain fluid retaining structure. The sample may be contacted with the backing element (i.e., deposited into a fluid retaining structure) using any suitable protocol, for instance, by pipette or other fluid dispenser. The resultant modified backing element supported sample may then be contacted with an array. To contact a modified backing element supported sample with the array element, the array element and backing element supported sample may be brought together in a manner sufficient so that the sample contacts the ligands of the array (as described above with reference to FIGS. 5A-5C). As such, the array may be placed on top of the treated backing element supported sample to produce an array assay chamber.

Following contact of the array and the sample, the resultant assay chamber of the array assay device is then maintained under conditions sufficient for any binding complexes between members of specific binding pairs to occur. Where desired, the sample may be agitated or mixed (e.g., using bubble mixing where a bubble has been provided in the array assay chamber) to ensure contact of the sample with the array. For instance, the assay chamber may be turned upside down to ensure that the sample contacts the entire array surface. In the case of hybridization assays, the modified backing element supported sample may be contacted with the microarray under stringent hybridization conditions, whereby complexes are formed between target nucleic acids that are complementary to probe sequences attached to the array surface, i.e., duplex nucleic acids are formed on the surface of the substrate by the interaction of the probe nucleic acid and its complement target nucleic acid present in the sample. The backing element supported sample may be incubated as desired prior to contact with an array or may be immediately contacted with an array assembly and incubated in conjunction therewith.

Once the incubation step is complete, the backing element and array element may be separated and the array may be washed one or more times to remove any unbound and non-specifically bound sample from the microarray assembly. Washing agents used in array assays may vary depending on the particular binding pair used in the particular assay. For example, in those embodiments employing nucleic acid hybridization, washing agents of interest include, but are not limited to: salt solutions such as sodium, sodium phosphate and sodium, sodium chloride and the like, at different concentrations and may include some surfactant as well. For example, an array may be washed in, first, 6×SSC with 0.005% Triton X102 at about 60° C. or at about 20° C. and then 0.1×SSC at about 20° C. or at about at about 4°.

Following the washing procedure, as described above, the array may then be interrogated or read so that the presence of the binding complexes may be detected, e.g., through use of a signal production system, e.g. an isotopic or fluorescent label present on the analyte, etc., as described above. The presence of the analyte in the sample may then be deduced from the detection of binding complexes on the microarray solid support (e.g., substrate) surface.

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, Internet, etc.

Accordingly, in use a modified backing element may be mated with a microarray assembly such that at least one gasket is positioned therebetween to provide an array assay chamber in which an array assay may be performed, wherein the array will may be exposed to a sample (for example, a fluorescently labeled analyte, e.g., protein containing sample) and the array then read.

Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere).

Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

### Kits

In one aspect of the invention, kits are provided. In certain embodiments, the subject kit includes at least one, e.g., 2 or more, 3 or more, 4 or more, 5 or more, etc., backing element produced in accordance with embodiments of the invention. The subject kit may also include one or more array elements.

A kit of the invention may also include a device for holding a backing element and array element in a fixed position relative to each other to perform an array assay, such as an array assay device holder that provides a compression force to at least one member of a backing element/array element of the array assay device. Array assay device holders of interest include, but are not limited to, those devices described in published United States Patent Application publication nos. 2003-0235518-A1; 2003-0235521-A1; 2003-0235520-A1 and 2003-0235825-A1; the disclosures of which are herein incorporated by reference.

The kit may further include one or more additional components necessary for carrying out an analyte detection assay, such as sample preparation reagents, buffers, labels, and the like. As such, a kit may include one or more containers such as vials or bottles, with each container containing a separate component for the assay, and reagents for carrying out an array assay such as a nucleic acid hybridization assay or the like. The kit may also include a denaturation reagent for denaturing an analyte, buffers such as hybridization buffers, wash mediums, enzyme substrates, reagents for generating a labeled target sample such as a labeled target nucleic acid sample, negative and positive controls.

In certain embodiments, a plurality of modified backing elements may be provided, where some or all may be the same or some or all may be different in one or more respects, e.g., differ in the number of fluid retaining structures present, the pattern of the one or more structures, the size of the one or more structures, the shape of the one or more structures, the material of the one or more structures, the volume of the one or more structures, etc., and/or differ in the size, shape, material, etc., of the modified backing element, etc., such that a variety of different modified backing elements may be provided in a kit for a variety of different applications and/or to fit with a variety of different microarrays.

In addition to the above components, the subject kits may also include instructions for using the components of the kit in an array assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g. via the Internet, are provided. An example of this embodiment is a kit that includes a web address where the instructions can be viewed and/or from which the instructions can be downloaded. As with the instructions, this means for obtaining the instructions is recorded on a suitable substrate.

In certain embodiments of the subject kits, the components of the kit are packaged in a kit containment element to make a single, easily handled unit, where the kit containment element, e.g., box or analogous structure, may or may not be an airtight container, e.g., to further preserve the one or more treated backing elements and one or more microarrays and reagents, if present, until use.

The following examples are offered by way of illustration and not by way of limitation.

### Experimental

One method of manufacturing backing elements includes securing a gasket to the surface of the planar rectangular solid support, followed by plasma treatment of the resultant structure and then a solvent soak of the resultant structure. Any impurities removed form the gasket element by the solvent soak are washed off of the backing element by a suitable wash. This procedure results in changes to the contact angle of the backing element from step to step. Also, the variability between backing elements processed in different batches at the various steps is significant.

The resultant variability of most concern is that of the last step which represents the product for utilization. This variability in contact angle is translated into the varying degrees of hydrophobicity/hydrophilicity experienced when applying a hybridization sample solution of the array assay. Hydrophobicity/hydrophilicity variations are problematic in terms of usability and thus functionality of the product. That is to say if the backing element is too hydrophilic (low contact angle) the hybridization sample solution spreads rapidly across the backing element surface and in some cases it “jumps” the gasket such that the hybridization sample solution can no longer be retained within the gasket element to properly conduct the array assay. If it is too hydrophobic (high contact angle) the hybridization sample solution does not spread beyond the point of application which can also result in the solution “jumping” the gasket or rather “splashing” as the backing and array elements are brought together. In either case the functionality of the array assay is compromised. A confounding issue to the way such backing elements are manufactured is the fact that the hydrophobicity/hydrophophilicity changes over time in a non-linear fashion. Thus, depending on the period between which the backing elements are manufactured to the time of use, the hydrophobicity/hydrophilicity would be significantly different. This variation in turn, affects the usability and ultimately the functionality of the array assay.

Functionality, therefore, is very important for an array assay and is addressed by this invention. This invention reverses the solvent/plasma steps in the protocol described above and eliminates the wash step. Employing this protocol reduces the variability of contact angle between batches, as compared to the protocol described above. However, the backing element's contact angle as produced in this protocol is very low after plasma treatment so an energy altering molecule, e.g., RainX, is applied to obtain the desired contact angle. This step ensures that the reproducibility of the contact angle between backing elements is high and stable over time. In turn, usability and functionality are less of a concern.

Altering the backing element contact angle was tested using a compound known in the commercial market as RainX. The backing elements, which were produced using the existing manufacturing procedure, were coated with various amounts (concentrations) of RainX. This coating step was done to determine what concentration would result in the approximate contact angle for the appropriate functionality of the backing element. Once the appropriate contact angle was determined a number of backing elements were coated to establish reproducibility. After applying a hybridization sample solution, the hydrophobicity/hydrophilicity was estimated by visual inspection as an approximation of the contact angle.

Establishing reproducibility of the contact angle was only the first step. The second step was to determine if coating the backing elements with such a compound as RainX would adversely affect the quality of the biological data generated via the array assay. To make that determination, 4 arrays were hybridized, 2 of which were coated with RainX and 2 were not coated. Of the 2 coated and 2 non-coated backing elements 1 was hybridized as a One-Color platform and the other as a Two-Color platform. The QC reports generated for each of the 4 arrays using Agilent Technologies, Inc. Feature Extraction software was examined. Comparisons were done between the coated and non-coated backing elements hybridized as a One-Color platform and between the coated and non-coated backing elements hybridized as a Two-Color platform.

The data comparison focused on the number of Feature Non-Uniformity Outliers, the Signal Distribution, the Percent CV of the replicated biological probes and the Spike-In Statistics. On all accounts the data between the rainX coated and non-coated backing elements were comparable. That is to say the data did not reveal any significant differences between the coated and non-coated backing elements. Therefore, the modified backing elements did not hinder the hybridization or the hybridization sample solution motion as suggested by the number of Feature Non-Uniformity Outliers, the Signal Distribution and the Percent CV of the replicated probes. The Spike-In statistics confirms that the rainX coating did not affect the data quality in either the One-Color or Two-Color platform.

Alternatively, the coating could be performed with n-decyl trichlorosilane by a chemical vapor deposition process on an MVD 100 deposition system (Applied MST) and a protocol recommended by the manufacturer, such as the one documented in the following publication (B. Kobrin and Jeff Chinn, “Vacuum-base technology for the deposition of self-assembled monolayers (SAMs)”, Invited paper: Russian Vacuum Technology Journal, December 2005. See the website produced by placing “[http://www.” before “appliedmst.com/pdf/references/russian_VacuumTechnology.pdf])”

All publications and patents cited in this specification are herein incorporated by reference as if each individual publication or patent were specifically and individually indicated to be incorporated by reference.

While the invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the invention. All such modifications are intended to be within the scope of the claims appended hereto.

What is claimed is:

1. A method for producing a backing element, the method comprising:
(a) providing a solid support having a fluid retaining structure on a planar surface thereof;
(b) contacting said solid support with a non-polar organic solvent, to produce a solvent contacted solid support;
(c) subjecting the solvent contacted solid support of (b) to a plasma to produce a plasma modified solid support; and
(d) contacting the plasma modified solid support of (c) with a surface energy modification agent to produce said backing element.

2. The method according to claim 1, wherein said non-polar organic solvent is selected from the group consisting of: hexane, heptane, toluene, benzene, xylene, cyclohexane, methyl, ethyl, glyme, diglyme and triglyme.

3. The method according to claim 1, wherein said contacting of said solid support with said solvent comprises soaking the solid support in said solvent.

4. The method according to claim 1, wherein said plasma is a plasma of nitrogen, air, argon, oxygen, nitrous oxide, helium, water vapor, carbon dioxide, methane, and combinations thereof.

5. The method according to claim 4, wherein said plasma is a plasma of oxygen.

6. The method according to claim 1, wherein said contacting of said solid support with said surface energy modification agent comprises chemical or molecular vapor deposition.

7. The method of claim 1, wherein said solid support further comprises a plurality of fluid retaining structures.

8. The method according to claim 1, wherein said method results in a change of surface energy of said solid support.

9. The method according to claim 8, wherein said surface energy is modified by being decreased.

10. The method according to claim 9, wherein said contacting of said surface with said surface energy modification agent produces a hydrophobic surface.

11. The method according to claim 1, wherein said surface energy of said backing element is measured so as to determine if the surface energy of said backing element is within a predetermined range.

12. The method according to claim 11, wherein said surface energy is determined by measuring the contact angle of a surface of the solid support.

13. The method according to claim 1, wherein said surface energy modification agent comprises a polysiloxane.

14. The method according to claim 13, wherein said polysiloxane is present in a fluid composition.

15. The method according to claim 14, wherein said fluid composition comprises a solvent.

16. The method according to claim 15, wherein said solvent comprises an alcohol.

17. The method according to claim 16, wherein said composition further comprises an acid.

18. The method according to claim 1, wherein said surface energy modification agent comprises a silane.

19. An array assay device comprising:
(a) a backing element produced according to the method of claim 1; and
(b) an array element.

20. The array assay device according to claim 19, further comprising a sample.

21. A method of detecting the presence of an analyte in a sample, said method comprising:
(a) placing said sample in an assay chamber of an array assay device according to claim 18; and
(b) detecting the presence of a binding complex on an array of said device to detect the presence of an analyte in said sample.

22. A kit comprising:
a backing element produced according to the method of claim 1; and
an array element.
